(19) United States
(12) Patent Application Publication
(10) Pub. No.: US 2022/0296268 A1
(43) Pub. Date: Sep. 22, 2022

(54) Device for the Removal and Loading of Surgical Blades and Methods of Use

(71) Applicant: Martien Laguna, Hoffman Estates, IL (US)

(72) Inventor: Martien Laguna, Hoffman Estates, IL (US)

(21) Appl. No.: 17/206,924

(22) Filed: Mar. 19, 2021

(51) Int. Cl. A61B 17/3213; A61B 17/00

(52) U.S. Cl. CPC A61B 17/3213; A61B 2017/0046

(57) ABSTRACT

The present disclosure provides a device for the loading and removal of a surgical blade. The device includes a base, a first sidewall extending vertically from a first side of the base, a second sidewall extending vertically from a second side of the base, and a third sidewall extending vertically from a first end of the base. The device also includes a first protrusion and a second protrusion each extending vertically from the base and positioned such that there is a gap between the first protrusion and the second protrusion at a second end of the base. The device also includes a moveable member coupled to the third sidewall and extending in a direction towards the second end of the base. The moveable member is movable from a first position to a second position in which a bottom surface of the moveable member is closer to the base.

### FIELD

The present invention relates generally to devices and methods for the loading and removal of a surgical blade from a scalpel handle.

### BACKGROUND

Unless otherwise indicated herein, the materials described in this section are not prior art to the claims in this application and are not admitted to be prior art by inclusion in this section.

Traditional scalpels that are used in a variety medical procedures include a scalpel handle with a removable surgical blade. Such an arrangement allows the surgical blade to be discarded after use, while the scalpel handle can be reused for multiple procedures. However, the act of loading and removing the surgical blade from the scalpel handle can result in injury. Traditionally, the surgical blade is loaded or removed from the scalpel handle using forceps. Such an arrangement does not always prevent injury since the sharp edge of the surgical blade is exposed during the loading and removal process. Therefore, an improved device for the loading and removal of a surgical blade from a surgical handle is desirable.

### SUMMARY

In a first aspect, the present disclosure provides a device comprising (a) a base including a first end, a second end opposite the first end, a first side, and a second side opposite the first side, (b) a first sidewall extending vertically from the first side of the base, (c) a second sidewall extending vertically from the second side of the base, (d) a third sidewall extending vertically from the first end of the base, (e) a first protrusion extending vertically from the base, wherein the first protrusion is positioned adjacent the second end of the base, (f) a second protrusion extending vertically from the base, wherein the second protrusion is positioned adjacent the second end of the base such that there is a gap between the first protrusion and the second protrusion at the second end of the base, and (g) a moveable member coupled to the third sidewall and extending in a direction towards the second end of the base, wherein the moveable member is movable from a first position to a second position, and wherein a bottom surface of the moveable member is closer to the base in the second position than in the first position.

In another aspect, the present disclosure provides a method comprising (a) positioning a surgical blade between the first sidewall and the second sidewall of the device of the first aspect such that a sharp end of the surgical blade is positioned at the first end of the base between the base and the moveable member, (b) transitioning the moveable member from the first position to the second position, wherein the surgical blade is pinned between the base and the bottom surface of the moveable member when the moveable member is in the second position, (c) positioning a scalpel handle between the first protrusion and the second protrusion and through a slot in the surgical blade in a direction towards the first end of the base until the scalpel handle is coupled to the surgical blade by a track system in the scalpel handle, (d) transitioning the moveable member from the second position to the first position, and (e) removing the coupled surgical blade and the scalpel handle from the device.

In yet another aspect, the present disclosure provides a method comprising (a) positioning a surgical blade that is coupled to a scalpel handle between the first sidewall and the second sidewall of the device of the first aspect such that a sharp end of the surgical blade is positioned at the first end of the base between the base and the moveable member, (b) transitioning the moveable member from the first position to the second position, wherein the surgical blade is pinned between the base and the bottom surface of the moveable member when the moveable member is in the second position, and (c) moving the scalpel handle in a direction away from the first end of the base until the scalpel handle separates from the surgical blade.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a side cross-sectional view of a device, according to an example embodiment.

FIG. 2 is a perspective view of the device of FIG. 1, according to an example embodiment.

FIG. 3 is a top view of the device of FIG. 1, according to an example embodiment.

FIG. 4 is a rear view of the device of FIG. 1, according to an example embodiment.

FIG. 5 is a flowchart of an example method, according to an example embodiment.

FIGS. 6A-6C illustrate a device carrying out the method steps of FIG. 5, according to an example embodiment.

FIG. 7 is a flowchart of another example method, according to an example embodiment.

FIGS. 8A-8C illustrate a device carrying out the method steps of FIG. 7, according to an example embodiment.

### DETAILED DESCRIPTION

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

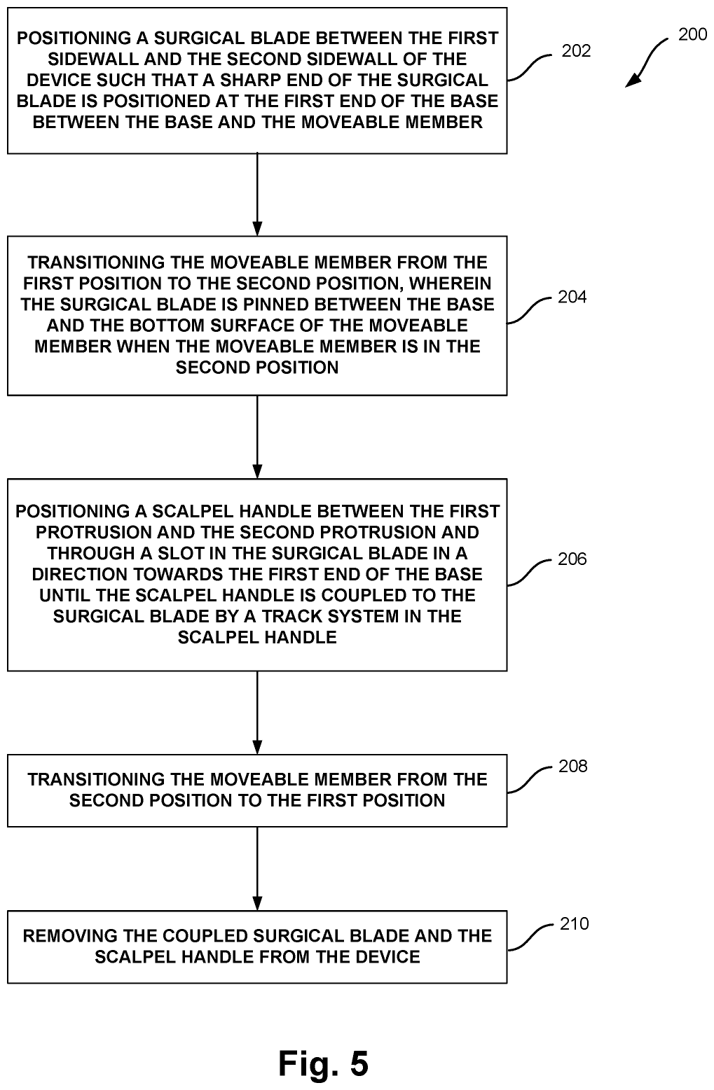

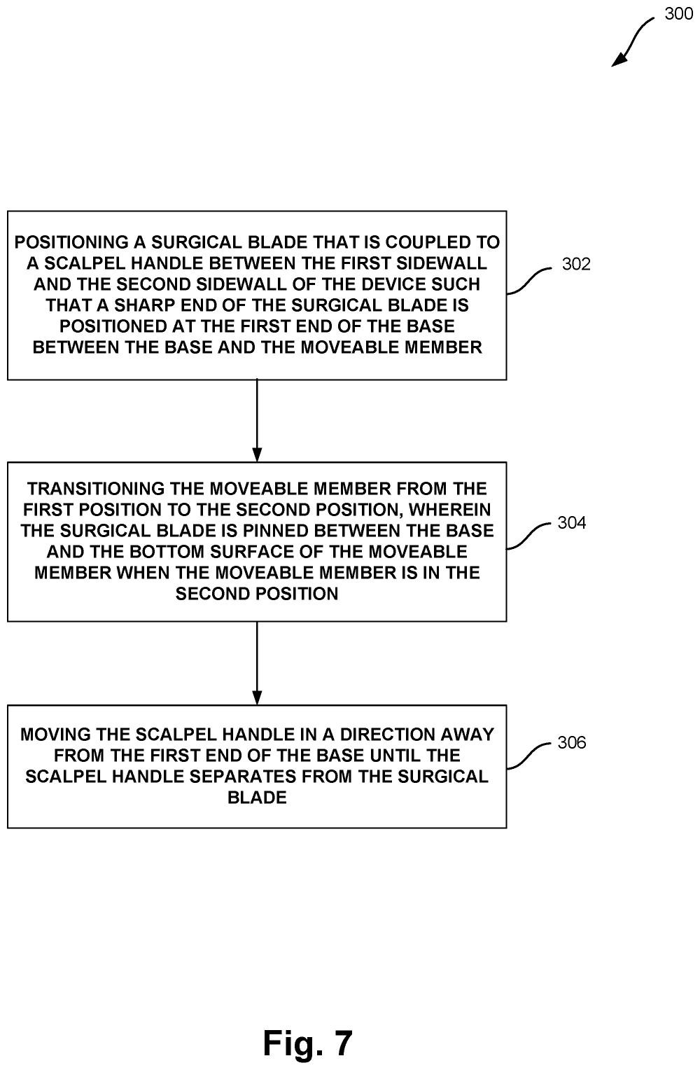

FIGS. 5 and 7 and the accompanying disclosure describing the operations of the methods set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Illustrative, non-exhaustive examples, which may or may not be claimed, of the subject matter according the present disclosure are provided below.

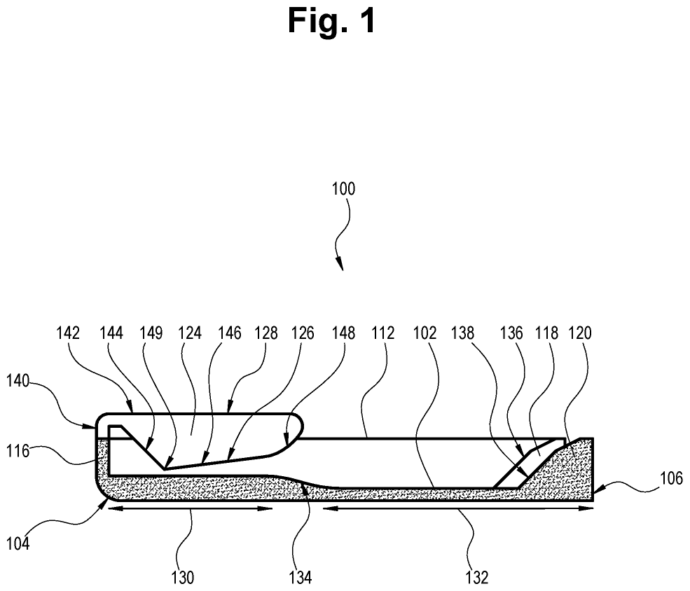

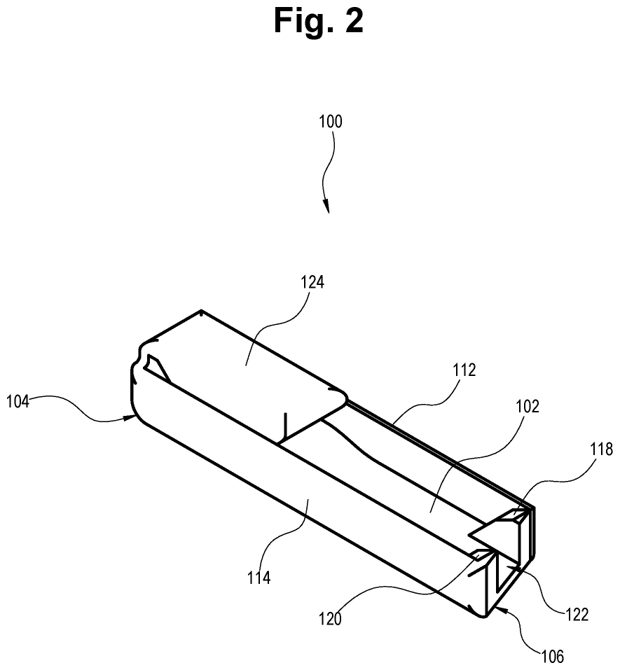

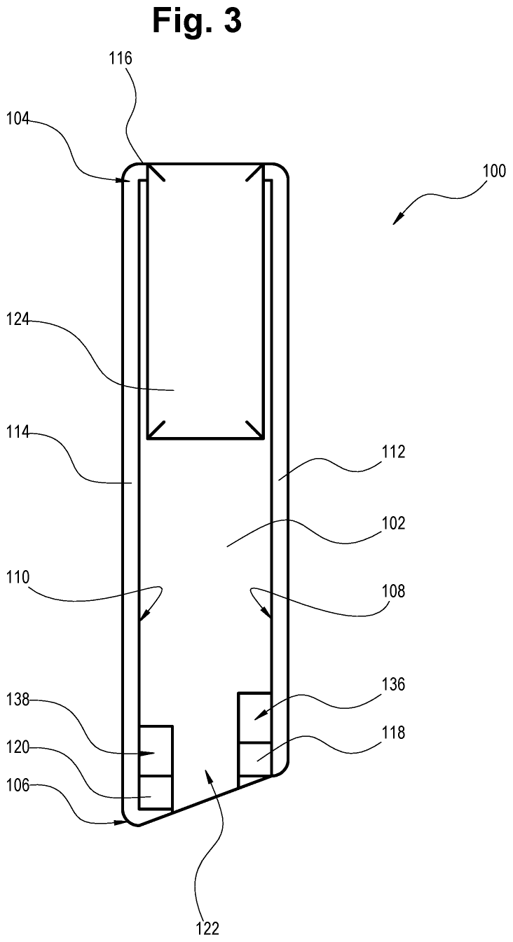

With reference to the Figures, FIG. 1 provides a side cross-sectional view of an example device 100. In particular, as shown in FIG. 1, the device 100 includes a base 102 including a first end 104 and a second end 106 opposite the first end 104. The base also includes a first side 108 and a second side 110 opposite the first side 108, as best shown in the top view of the device 100 of FIG. 3. The device 100 further includes a first sidewall 112 extending vertically from the first side 108 of the base 102, a second sidewall 114 extending vertically from the second side 110 of the base 102, and a third sidewall 116 extending vertically from the first end 104 of the base 102. FIG. 1 is a side cross-sectional view of the device 100 with the second sidewall 114 removed. The second sidewall 114 can be seen in the perspective view of the device 100 shown in FIG. 2.

The device 100 further includes a first protrusion 118 extending vertically from the base 102. As shown in top view of the device 100 in FIG. 3, the first protrusion 118 is positioned adjacent the second end 106 of the base 102. The device 100 further includes a second protrusion 120 extending vertically from the base 102. As shown in FIG. 3, the second protrusion 120 is positioned adjacent the second end 106 of the base 102 such that there is a gap 122 between the first protrusion 118 and the second protrusion 120 at the second end 106 of the base 102. The device 100 further includes a moveable member 124 coupled to the third sidewall 116 and extending in a direction towards the second end 106 of the base 102. The moveable member is movable from a first position to a second position, where a bottom surface 126 of the moveable member 124 is closer to the base 102 in the second position than in the first position.

FIG. 1 illustrates the moveable member 124 in the first position, which may also be referred to as a relaxed or default positon. As such, the first position is the position the moveable member 124 naturally assumes in the absence of any force applied to the top surface 128 of the moveable member 124. As shown in FIG. 1, in one example the bottom surface 126 of the moveable member 124 and the base 102 do not touch one another when the moveable member 124 is in the first position.

As shown in FIG. 1, in one example the base 102 includes a first portion 130 adjacent the first end 104 and a second portion 132 adjacent the second end 106. The relative thicknesses of the first portion 130 and the second portion 132 may be different. In one particular example, as shown in FIG. 1, a thickness of the first portion 130 is greater than a thickness of the second portion 132. Such an arrangement may enable easier loading of a surgical blade into the device 100 while reducing a distance between the bottom surface 126 of the moveable member 124 and the base 102. In one example, a length of the first portion 130 is approximately equal to a length of the moveable member 124. As further shown in FIG. 1, in one example an angled transition 134 is positioned between the first portion 130 and the second portion 132 of the base 102. Such an arrangement may enable easier loading of a surgical blade into the space between the bottom surface 126 of the moveable member 124 and the base 102.

In one example, a height of the first protrusion 118 and a height of the second protrusion 120 are equal, and a height of the first end 104 of the base 102 is less than the height of the first protrusion 118 and the height of the second protrusion 120. In such an arrangement, a surgical blade positioned in the device 100 has a resting position in which the sharp end of the surgical blade sits lower than the non-sharp side of the surgical blade. This serves to facilitate in easily matching a track system on the scalpel handle to the interior edges of the surgical blade where the scalpel handle is loaded.

As illustrated in FIG. 3, in one example a distance from the first protrusion 118 to the first end 104 of the base 102 is different than a distance from the second protrusion 120 to the first end 104 of the base 102. In the arrangement shown in FIG. 3, the distance from the first protrusion 118 to the first end 104 of the base 102 is less than the distance from the second protrusion 120 to the first end 104 of the base 102. Alternatively, in another example the distance from the first protrusion 118 to the first end 104 of the base 102 is greater than the distance from the second protrusion 120 to the first end 104 of the base 102. These configurations provide an angled second end 106 of the base 102. Such arrangements may be beneficial to match an angled end of the surgical blade that is configured to be positioned in the device 100 when in use. In another example, as shown in FIG. 2, a distance from the first protrusion 118 to the first end 104 of the base 102 is the same as a distance from the second protrusion 120 to the first end 104 of the base 102.

As shown in FIG. 1, in one example the first protrusion 118 includes a first angled surface 136 in a direction towards the first end 104 of the base 102, and the second protrusion 120 includes a second angled surface 138 in a direction towards the first end 104 of the base 102. In use, the non-sharp side of a surgical blade can rest on the first angled surface 136 and the second angled surface 138. As discussed above, the height of the first angled surface 136 and the second angled surface 138 is greater than the height of the first end 104 of the base 102 such that the non-sharp side of the surgical blade sits higher than the sharp end of the surgical blade to facilitate in easily matching a track system on the scalpel handle to the interior edges of the surgical blade where the scalpel handle is loaded.

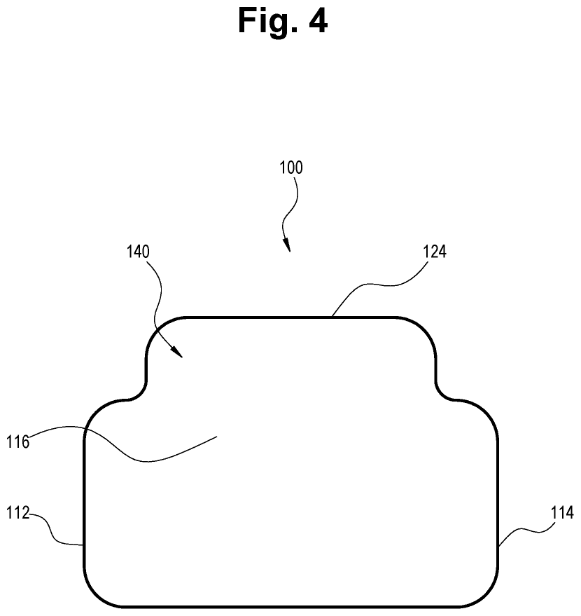

FIG. 4 illustrates a rear view of the device 100. As shown in FIG. 4, a width of the moveable member 124 is less than a distance between the first sidewall 112 and the second sidewall 114. Such an arrangement enables the moveable member 124 to move down towards the base 102 when the moveable member 124 transitions from the first position to the second position.

As shown in FIG. 1, the moveable member 124 may comprise a first portion 140 extending vertically from the third sidewall 116 and a second portion 142 extending in a direction towards the second end 106 of the base 102. As such, the moveable member 124 may comprise a cantilever configured to transition from the first position to the second position in response to a force applied to the top surface 128 of the moveable member 124. As further shown in FIG. 1, the bottom surface 126 of the moveable member 124 may have a wedge shape. In particular, the bottom surface 126 of the moveable member 124 may include a first angled portion 144 and a second angled portion 146. The first angled portion 144 may have a greater (e.g., steeper) angle with respect to the base 102, while the second angled portion 146 may have a smaller (e.g., more gradual) angle with respect to the base. Further, as shown in FIG. 1, the end of the moveable member nearest the second end 106 of the base may include a rounded edge 148. The first angled portion 144 and second angled portion 146 may meet at an edge 149. The edge 149 represents the portion of the bottom surface 126 of the moveable member 124 that is closest to the base 102. The arrangements described above enables easier loading of the surgical blade into the space between the bottom surface 126 of the moveable member 124 and the base 102, while minimizing a distance the moveable member 124 must travel to contact a surgical blade positioned therein.

In one example, the gap 122 between the first protrusion 118 and the second protrusion 120 is about 4 mm. In another example, a length of the base 102 ranges from about 37 mm to about 40 mm. In another example, a height of the first sidewall 112 and a height of the second sidewall 114 is about 5 mm. In another example, a distance between the first sidewall 112 and the second sidewall 114 is about 8 mm. These dimension are provided for illustrated purposes, and other dimensions are possible as well to operate the device 100 with a variety of surgical blades and corresponding scalpel handles.

In one example, the entire device 100 comprises a single material. For example, the device may be made from a machinable plastic material such as polyether ether ketone (PEEK), polyethylene (PE), polypropylene (PP), or polyoxymethylene (POM). In another example, the first portion 130 of the base 102 and/or the bottom surface 126 of the moveable member 124 may comprise a material that is different from the rest of the device 100. In one particular example, the first portion 130 of the base 102 and/or the bottom surface 126 of the moveable member 124 may comprise a material that has a greater coefficient of friction than the coefficient of friction of the rest of the device 100. In one particular example, the first portion 130 of the base 102 and/or the bottom surface 126 of the moveable member 124 may comprise a rubber material, such as silicone rubber as a non-limiting example.

In some examples, such as shown in any one of FIGS. 1-4, one or more components of the device 100 is made via an additive manufacturing process using an additive-manufacturing machine, such as stereolithography, multi jet modeling, inkjet printing, selective laser sintering/melting, and fused filament fabrication, among other possibilities. Additive manufacturing enables one or more components of the device 100 and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the device 100 using a Computer Aided Design (CAD) of the device 100 as instructions. As a result, changes to the design of the device 100 can be immediately carried out in subsequent physical creations of the device 100. This enables the components of the device 100 to be easily adjusted or scaled to fit different types of applications (e.g., for use with various types and sizes of surgical blades and scalpel handles).

The layer-upon-layer process utilized in additive manufacturing can deposit one or more components of the device 100 with complex designs that might not be possible for devices assembled with traditional manufacturing. In turn, the design of the device 100 can include aspects that aim to improve overall operation. For example, the design can incorporate physical elements that help redirect stresses in a desired manner that traditionally manufactured devices might not be able to replicate.

Additive manufacturing also enables depositing one or more components of the device 100 in a variety of materials using a multi-material additive-manufacturing process. In such an example, the majority of the device 100 may be made from a first material and the first portion 130 of the base 102 and/or the bottom surface 126 of the moveable member 124 may be made from a second material that is different than the first material. In one particular example, the first material comprises a plastic such as polyether ether ketone (PEEK), polyethylene (PE), polypropylene (PP), or polyoxymethylene (POM), and the second material comprises a high friction material such as rubber as a non-limiting example. In another example, both the entire device 100 is made from the same material. Other example material combinations are possible as well. Further, one or more components of the device 100 can have some layers that are created using a first type of material and other layers that are created using a second type of material.

In use, the device 100 provides a surgical apparatus that allows the user to safely and swiftly load and unload a surgical blade from a variety of scalpel handles (such as a short and long #3 scalpel handle as non-limiting examples). The sharp end of the surgical blade rests between the bottom surface 126 of the moveable member 124 and the base 102. The non-sharp end of the surgical blade rests on top of the first protrusion 118 and the second protrusion 120, which are spaced apart from each other to form a gap 122 in order to slide the scalpel handle in and out of the device 100 for loading and unloading of the surgical blade. The first protrusion 118 and the second protrusion 120 provide the leverage needed to pry the scalpel handle away from the surgical blade by applying a downward motion, thereby leaving the surgical blade in place but separating the scalpel handle away from the surgical blade. Once decoupled, the scalpel handle is moved away from the surgical blade and device 100 to complete the process of unloading the surgical blade.

To aid in the loading and unloading of the surgical blade to the scalpel handle, the bottom surface 126 of the moveable member 124 is used to clamp down the top portion of the surgical blade on the sharp side against the base 102. This creates a strong friction force that prevents the surgical blade from moving or sliding away while being loaded and unloaded from the scalpel handle. In addition, the clamping feature of the bottom surface 126 of the moveable member 124 protects the user from any of the sharp aspects of the surgical blade by enclosing the sharp end of the surgical blade within the sidewalls of the device 100. As discussed above, the first and second protrusions 118, 120 and the base 102 are constructed with staggered heights with the first and second protrusions 118, 120 sitting slightly taller than the height of the base when the device 100 is laid flat. This arrangement creates an angled resting position of the surgical blade where the sharp end of the surgical blade sits lower than the non-sharp side of the surgical blade, which facilitates the easily matching of a track system on the scalpel handle to the interior edges of the surgical blade where the scalpel handle is loaded.

FIG. 5 is a block diagram of an example of a method 200 for loading a surgical blade onto a scalpel handle. Method 200 shown in FIG. 5 presents an embodiment of a method that could be used with any of the embodiments of the device 100 described above in relation to FIGS. 1-4, as an example. Method 200 includes one or more operations, functions, and/or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 202, the method 200 includes positioning a surgical blade between the first sidewall 112 and the second sidewall 114 of the device 100 such that a sharp end of the surgical blade is positioned at the first end 104 of the base 102 between the base 102 and the moveable member 124.

At block 204, the method 200 includes transitioning the moveable member 124 from the first position to the second position, where the surgical blade is pinned between the base 102 and the bottom surface 126 of the moveable member 124 when the moveable member 124 is in the second position. This step may be accomplished by applying a force to the top surface 128 of the moveable member 124 (e.g., pinching the moveable member 124).

At block 206, the method 200 includes positioning a scalpel handle between the first protrusion 118 and the second protrusion 120 and through a slot in the surgical blade in a direction towards the first end 104 of the base 102 until the scalpel handle is coupled to the surgical blade by a track system in the scalpel handle.

At block 208, the method 200 includes transitioning the moveable member 124 from the second position to the first position. This step may be accomplished by applying simply removing the force from the top surface 128 of the moveable member 124, and enabling the moveable member 124 to spring back to the first position due to the material properties of the device 100.

At block 210, the method 200 includes removing the coupled surgical blade and the scalpel handle from the device 100.

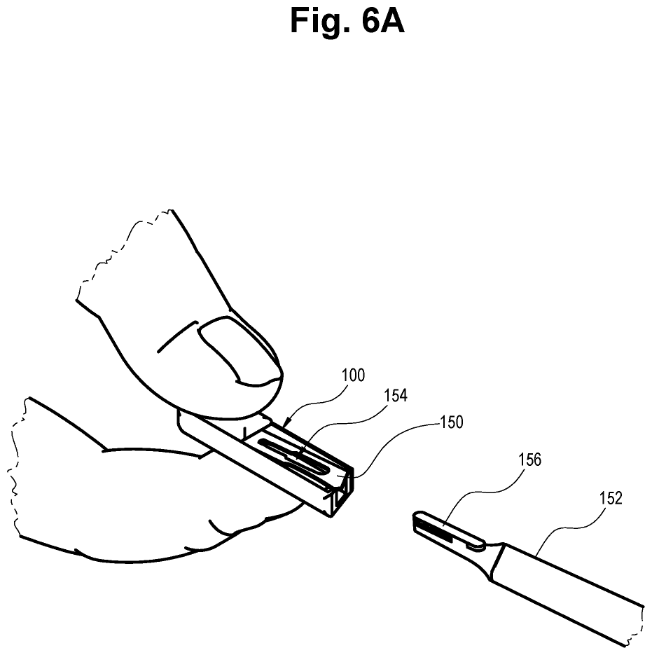

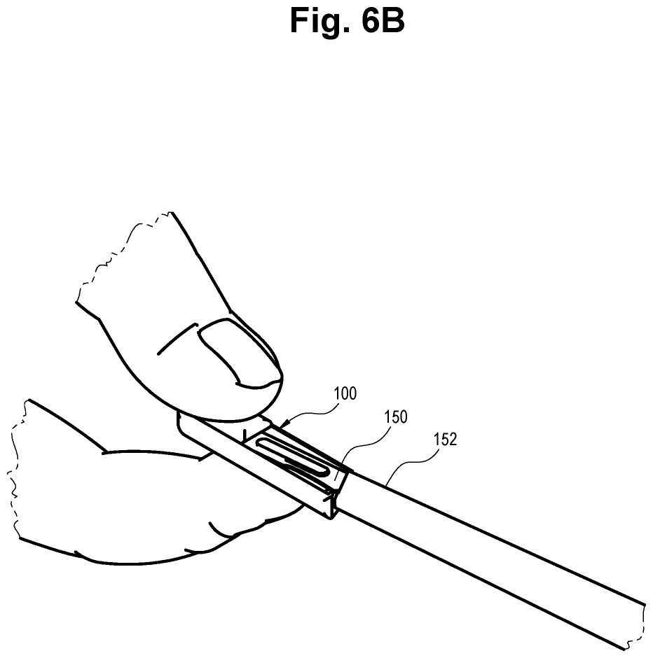

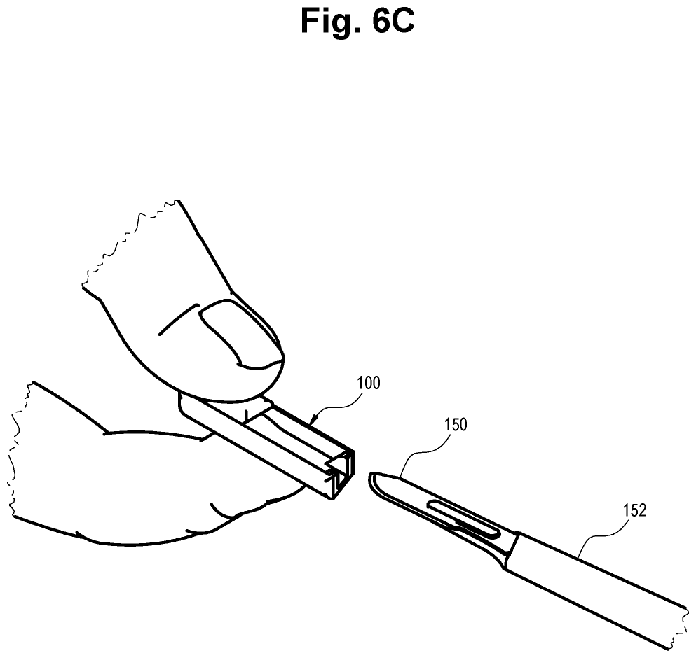

FIGS. 6A-6C illustrate the method steps 202-210 discussed above. In particular, FIG. 6A illustrates the surgical blade 150 positioned between the first sidewall 112 and the second sidewall 114 of the device 100. FIG. 6A further shows the moveable member 124 in the second position such that the surgical blade 150 is pinned between the base 102 and the bottom surface 126 of the moveable member 124. FIG. 6B illustrates the scalpel handle 152 positioned between first protrusion 118 and the second protrusion 120 and through a slot 154 in the surgical blade 150 in a direction towards the first end 104 of the base 102 until the scalpel handle 152 is coupled to the surgical blade 150 by a track system 156 in the scalpel handle 152. FIG. 6C illustrates the coupled surgical blade 150 and the scalpel handle 152 being removed from the device 100 after the moveable member 124 is transitioned from the second position to the first position.

FIG. 7 is a block diagram of an example of a method 300 for unloading a surgical blade from a scalpel handle. Method 300 shown in FIG. 7 presents an embodiment of a method that could be used with device 100 described above in relation to FIGS. 1-4, as an example. Method 300 includes one or more operations, functions, and/or actions as illustrated by one or more of blocks 302-306, which when performed in concert can be considered a dispensing session. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 302, the method 300 includes positioning a surgical blade that is coupled to a scalpel handle between the first sidewall 112 and the second sidewall 114 of the device 100 such that a sharp end of the surgical blade is positioned at the first end 104 of the base 102 between the base 102 and the moveable member 124.

At block 304, the method 300 includes transitioning the moveable member 124 from the first position to the second position, where the surgical blade is pinned between the base 102 and the bottom surface 126 of the moveable member 124 when the moveable member 124 is in the second position.

At block 306, the method 300 includes moving the scalpel handle in a direction away from the first end 104 of the base 102 until the scalpel handle separates from the surgical blade.

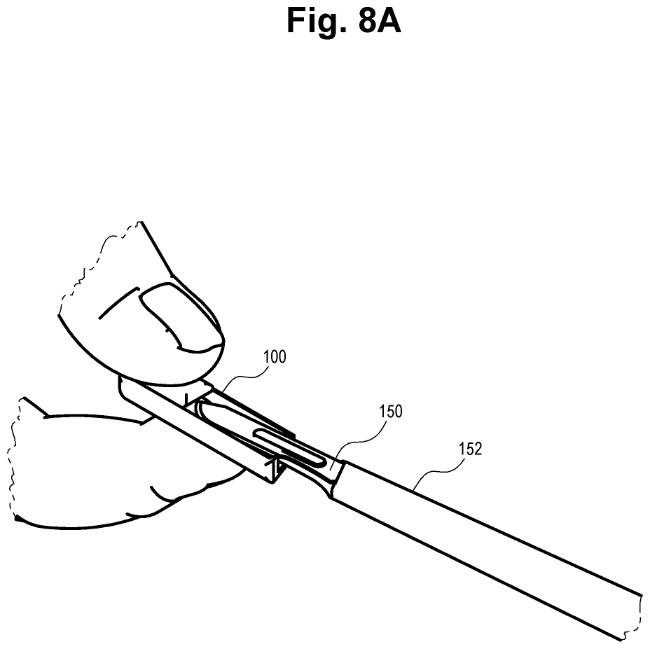

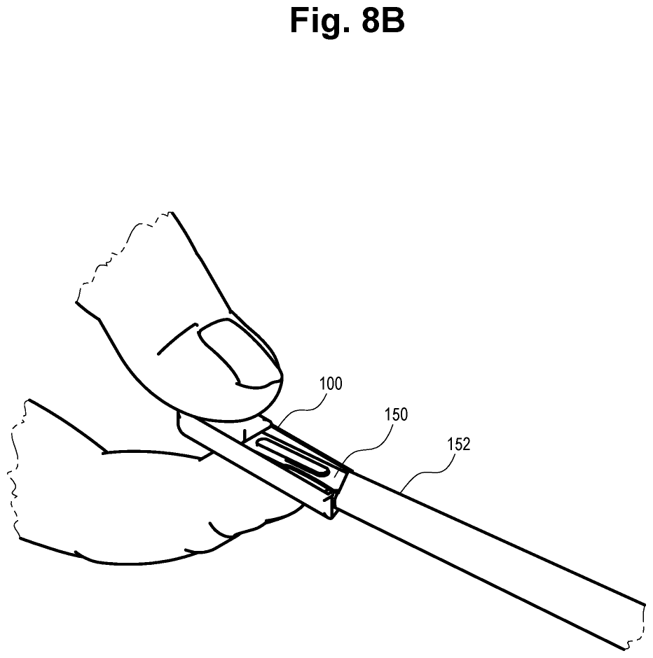

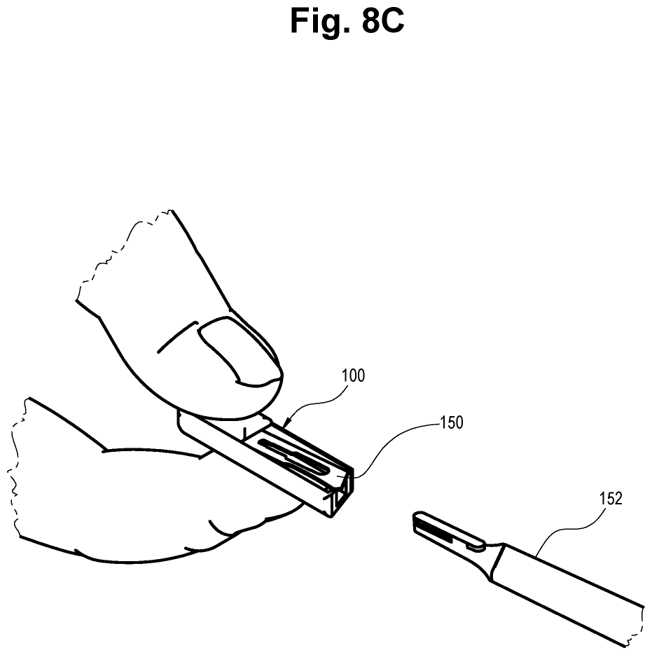

FIGS. 8A-8C illustrate the method steps 302-306 discussed above. In particular, FIG. 8A illustrates the coupled surgical blade 150 and scalpel handle 152 positioned between the first sidewall 112 and the second sidewall 114 of the device 100. FIG. 8B illustrates the moveable member 124 transitioning from the first position to the second position such that the surgical blade 150 is pinned between the base 102 and the bottom surface 126 of the moveable member 124. FIG. 8C illustrates the scalpel handle 152 moving in a direction away from the first end 104 of the base 102 until the scalpel handle 152 separates from the surgical blade 150.

The methods described herein can be utilized effectively with any of the examples or variations of the devices and systems described above, as well as with other examples and variations not described explicitly in this document. The features of any of the systems, devices, or components thereof described in any of the examples herein can be used in any other suitable example of a device or device component.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

1. A device comprising:
a base including a first end, a second end opposite the first end, a first side, and a second side opposite the first side;
a first sidewall extending vertically from the first side of the base;
a second sidewall extending vertically from the second side of the base;
a third sidewall extending vertically from the first end of the base;
a first protrusion extending vertically from the base, wherein the first protrusion is positioned adjacent the second end of the base;
a second protrusion extending vertically from the base, wherein the second protrusion is positioned adjacent the second end of the base such that there is a gap between the first protrusion and the second protrusion at the second end of the base; and
a moveable member coupled to the third sidewall and extending in a direction towards the second end of the base, wherein the moveable member is movable from a first position to a second position, and wherein a bottom surface of the moveable member is closer to the base in the second position than in the first position.

2. The device of claim 1, wherein the bottom surface of the moveable member and the base do not touch one another when the moveable member is in the first position.

3. The device of claim 1, wherein the base includes a first portion adjacent the first end and a second portion adjacent the second end, and wherein a thickness of the first portion is greater than a thickness of the second portion.

4. The device of claim 3, wherein a length of the first portion is approximately equal to a length of the moveable member.

5. The device of claim 3, wherein an angled transition is positioned between the first portion and the second portion of the base.

6. The device of claim 1, wherein a height of the first protrusion and a height of the second protrusion are greater than a height of the first end of the base.

7. The device of claim 1, wherein a distance from the first protrusion to the first end of the base is different than a distance from the second protrusion to the first end of the base.

8. The device of claim 1, wherein the first protrusion includes a first angled surface in a direction towards the first end of the base, and wherein the second protrusion includes a second angled surface in a direction towards the first end of the base.

9. The device of claim 1, wherein a width of the moveable member is less than a distance between the first sidewall and the second sidewall.

10. The device of claim 1, wherein the moveable member comprises a first portion extending vertically from the third sidewall and a second portion extending in a direction towards the second end of the base.

11. The device of claim 1, wherein the bottom surface of the moveable member has a wedge shape.

12. The device of claim 1, wherein the moveable member comprises a cantilever.

13. The device of claim 1, wherein the gap between the first protrusion and the second protrusion is about 4 mm.

14. The device of claim 1, wherein a length of the base ranges from about 37 mm to about 40 mm.

15. The device of claim 1, wherein a height of the first sidewall and a height of the second sidewall is about 5 mm.

16. The device of claim 1, wherein a distance between the first sidewall and the second sidewall is about 8 mm.

17. A method comprising:
positioning a surgical blade between the first sidewall and the second sidewall of the device of claim 1 such that a sharp end of the surgical blade is positioned at the first end of the base between the base and the moveable member;
transitioning the moveable member from the first position to the second position, wherein the surgical blade is pinned between the base and the bottom surface of the moveable member when the moveable member is in the second position;
positioning a scalpel handle between the first protrusion and the second protrusion and through a slot in the surgical blade in a direction towards the first end of the base until the scalpel handle is coupled to the surgical blade by a track system in the scalpel handle;
transitioning the moveable member from the second position to the first position; and
removing the coupled surgical blade and the scalpel handle from the device.

18. A method comprising:
positioning a surgical blade that is coupled to a scalpel handle between the first sidewall and the second sidewall of the device of claim 1 such that a sharp end of the surgical blade is positioned at the first end of the base between the base and the moveable member;
transitioning the moveable member from the first position to the second position, wherein the surgical blade is pinned between the base and the bottom surface of the moveable member when the moveable member is in the second position; and
moving the scalpel handle in a direction away from the first end of the base until the scalpel handle separates from the surgical blade.
